(19) United States
(12) Patent Application Publication
(10) Pub. No.: US 2004/0014808 A1
(43) Pub. Date: Jan. 22, 2004

(54) Novel derivatives of carnosic acid

(76) Inventors: John P.N. Rosazza, Iowa City, IA (US); Mohammed Hosny, Makrum Abid-Nacer City (EG); Holly Ann Johnson, West Des Moines, IA (US)

Correspondence Address: DAVIS, BROWN, KOEHN, SHORS & ROBERTS, P.C., THE FINANCIAL CENTER, 666 WALNUT STREET, SUITE 2500, DES MOINES, IA, 50309-3993, US

(21) Appl. No.: 10/409,204

(22) Filed: Apr. 8, 2003

Related U.S. Application Data
(60) Provisional application No. 60/371,019, filed on Apr. 9, 2002.

(51) Int. Cl. A61K 31/335; A61K 31/337; C07C 61/29; C07D 303/06

(52) U.S. Cl. 514/475; 514/569; 514/732; 549/544; 562/404; 568/733

(57) ABSTRACT

Incubations with Nocardia sp., NRRL 5646 were conducted to produce new derivatives of the abietane-diterpene chemoprotectant and antioxidant, carnosic acid. Reduction of the C-20 carboxylic acid functional group followed by methylation at the C-12 phenol afforded the novel compound 11,20-dihydroxy-12-methoxy-abiet-8,11,13-triene. Oxidative cyclization of carnosic acid to carnosol followed by dihydroxylation at the isopropyl moiety afforded the novel compound 11,12,16,17-tetrahydroxy-7-10-(epoxymethano)-abiet-8,11,13-triene-20-one. The metabolites are new carnosic acid derivatives whose structures were confirmed by mass spectrometry and NMR spectroscopic analysis. The radical quenching properties of the new metabolites using the 2,2-diphenyl-1-picrylhydrazyl (DPPH) free-radical scavenging assay showed activities improved over that of mixed tocopherols and carnosic acid.

This application claims priority to U.S. Provisional Application Serial No. 60/371,019, filed Apr. 9, 2002.

### BACKGROUND OF THE INVENTION

The invention relates generally to novel compounds that are derivatives of carnosic acid and, more specifically, to novel compounds produced by the bioconversion of carnosic acid by Nocardia sp.

Diterpenes play diverse functional roles in plants, acting as hormones (gibberllins), photosynthetic pigments (phytol), and regulators of wound-induced responses (abietic acid) (Hanson, R. Nat. Prod. Reports 1995, 12, 207-218). Carnosic acid is an abietane-diterpene chemoprotectant that is produced by many plants, particularly plants of the family Lamiaceae, in particular, rosemary (Rosemarinus officinalis) and sage (Curvelier, M. E.; Richard, H.; Berset, C. J. Am. Oil Chem. Soc. 1996, 73, 645-652; Loliger, J. The Use of Antioxidants In Food. In Free Radicals and Food Additives; Aruoma, O. I.; Halliwell, B. Eds.; Taylor and Francis: London, 1991; Chapter 6). Carnosic acid is a precursor of many other related diterpenes (Luies, J. G.; Quinones, W.; Grilo, T. A.; Kishi, M. P. Phytochemistry 1994, 35, 1371-1374) including carnosol and both of these compounds are powerful antioxidants (Logliger, supra; Offord, E. A.; Guillot, F.; Aeschbach, R.; Loliger, J.; Pfeifer, A. M. A. Antioxidant and Biological Properties of Rosemary Components: Implications for Food and Health, In Natural Antioxidants: Chemistry, Health Effects, and Application, Shahidi, F. Ed.; AOCS Press, Champaign, Ill., 1997, pp. 88-96). The o-diphenol (catechol) structure of carnosic acid is responsible for its ability to inhibit lipid peroxidation and superoxide generation in isolated chloroplasts and microsomes versus chemically induced oxidative stresses (Munné-Bosch, S.; Schwarz, K.; Alegre, L. Free Rad. Res. 1999, 31, S107-112).

Mauda et al. (Masuda, T.; Inaba, Y.; Takeda, Y. J. Agric. Food Chem. 2001, 49, 5560-5565) suggested that hydrogen donation from the 11-position phenol to radical species, such as lipid peroxy radical, was involved in carnosic acid's antioxidant activity. Antioxidant properties of carnosic acid may explain the modulation of drug metabolizing enzymes involved in carcinogen activation and detoxification (Aruoma, O. I.; Halliwell, B.; Aeschbach, R.; Loliger, J. Xenobiotica 1992, 22, 257-268). Carnosic acid and carnosol inhibit cytochrome P450 activation of carcinogens in human cells in vitro (Offord, E. A.; Mace, K.; Ruffieux, C.; Malone, A.; Pfeifer, A. M. Carcinogenesis 1995, 16, 2057-2062) and enhance the activities of conjugating enzymes involved in carcinogen detoxification pathways in vivo (Singletary, K. W. Cancer Letters 1996, 100, 139-144). Moreover, carnosic acid enhances gene expression consistent with 1α-25-deihydroxyvitamin D₃, all-trans-retinoic acid or 12-O-tetradecanoylphorbol-13-acetate induced monocytic differentiation of HL-60-G cells, resulting in decreased cell proliferation and blocking cell cycle transition from G₁ to S phase (Danilenko, M.; Wang, X.; Studzinski, G. P. J. Natl. Cancer Inst. 2001, 93, 1224-123).

Worldwide demand for natural antioxidants has been rising due to safety concerns about synthetic food and feed additives and the public perception that natural food and feed supplements provide certain health benefits. The most important natural antioxidants being exploited commercially today are tocopherols. Carnosic acid has antioxidant properties similar to mixed tocopherols and its use is increasing.

It is well recognized that the effectiveness of particular antioxidant compound varies substantially from food to food or substrate to substrate. Further, the effectiveness of any particular compound cannot be accurately predicted for new foods or substrates. There is always a demand, accordingly, for new compounds with antioxidant activity that may be used either individually or in combination with existing antioxidants.

Nocardia sp. NRRL 5646 contains an astonishing array of enzymes many of which are similar to those found in mammalians. A soluble form of nitric oxide synthase (NOS_{NOC}) (Chen, Y,; Rosazza, J. P. N. J. Bacteriol. 1995, 177, 5122-5128), carboxylic acid reductase (Li, T.; Rosazza, J. P. N. Appl. Environ. Microbiol. 2000, 66, 684-687), aryl aldehyde oxidoreductase (Li, T.; Rosazza, J. P. N. J. Bacteriol. 1997, 179, 3482-3487), aldehyde reductase (Li, T.; Rosazza, J. P. N. J. md. Micrbiol. Biotech. 2000, 25, 328-332), guanylate cyclasel (Masuda, T.; Inaba, Y.; Takeda, Y. J. Agric. Food Chem. 2001, 49, 5560-5565), and ornithine transcarbamoylasel (Aruoma, O. I.; Halliwell, B.; Aeschbach, R.; Loliger, J. Xenobiotica 1992, 22, 257-268), have all been characterized in this organism. Activities for Cyclohydrolase I, sepiapterin reductase, vinylphenol hydratase (Farid, I. S. Arginine Biosynthesis in Nocardia NRRL 5646 and iNOS Inhibition by Noformycin and its Analogs. Ph.D. Thesis, The University of Iowa, Iowa City, Iowa, 2001 pp 43-82), hydroxyethylphenol oxidoreductase (Lee, K. S.; Rosazza, J. P. N. Biocatalytic oxidation of 4-vinylphenol by Nocardia. (2002) Can. J. Chem. (80) 582-588) and vanillate decarboxylase are under investigation in our laboratory. Because of these qualities, Nocardia was selected for the bioconversion of carnosic acid in an attempt to produce novel compounds.

### SUMMARY OF THE INVENTION

The invention consists of novel compounds that are derivatives of carnosic acid produced through the bioconversion of carnosic acid by Nocardia sp. Carnosic acid is applied to cultures of Nocardia and incubated. The products are removed and isolated.

Analysis of the products revealed three derivatives of carnosic acid with substantial yield, the new compound 11,20-dihydroxy-12-methoxy-abiet-8,11,13-triene, the known compound carnosol (11,20-dihydroxy-7-10-(epoxymethano)-abiet-8,11,13-triene-20-one), and the new compound 11,12,16,17-tetrahydroxy-7-10-(epoxymethano)-abiet-8,11,13-triene-20-one.

The two new compounds have antioxidant properties higher than that of carnosic acid.

An object of the present invention is to provide novel compounds.

Another object of the invention is to provide novel compounds that are derivatives of carnosic acid.

A further object of the invention is to provide novel compounds that have antioxidant activity.

Yet another object of the invention is to provide novel compounds produced through the bioconversion of carnosic acid by Nocardia sp.

These and other objects will be made apparent to those skilled in the art upon a review of this specification and the appended claims.

### DETAILED DESCRIPTION OF A PREFERRED EMBODIMENT

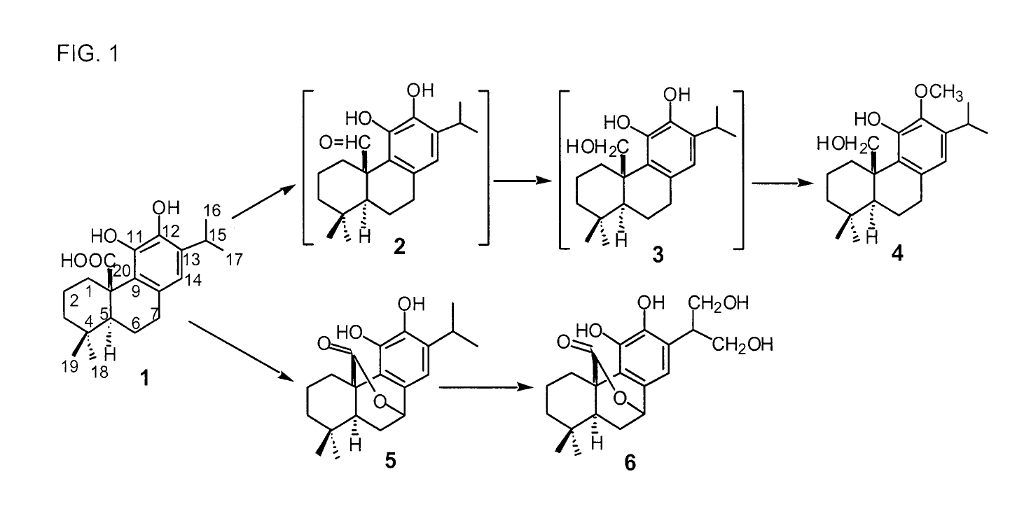

The invention relates to the ncubation with Nocardia sp., NRRL 5646 of the abietane-diterpene chemoprotectant and antioxidant, carnosic acid (Compound 1 in FIG. 1), to produce derivatives. Reductive biotransformation of the C-20 carboxylic acid functional group followed by biological methylation at the C-12 phenol afforded the novel compound 4, having the name 11,20-dihydroxy-12-methoxy-abiet-8,11,13-triene. Oxidative cyclization of carnosic acid to carnosol (Compound 5 in FIG. 1) followed by dihydroxylation at the isopropyl moiety afforded the novel compound 6, having the name 11,12,16,17-tetrahydroxy-7-10-(epoxymethano)-abiet-8,11,13-triene-20-one. The metabolites are new carnosic acid derivatives whose structures were confirmed by mass spectrometry and NMR spectroscopic analysis. The radical quenching properties of the new metabolites using the 2,2-diphenyl-1-picrylhydrazyl (DPPH) free-radical scavenging assay showed activities improved over that of mixed tocopherols and carnosic acid.

Nocardia sp. NRRL 5646 reproducibly forms three major metabolites of carnosic acid 1 in good yield. Of these, two were previously unknown carnosic acid derivatives Compound 4 and Compound 6, and one was the known natural product carnosol 5. None of the observed metabolites were formed in control cultures or in media containing no microorganisms but incubated under the same conditions. Because of the antioxidant characteristics of carnosic acid embodied in the catechol moiety, it is surprising that carnosic acid did not spontaneously form Compound 5 or 7α-hydroxycarnosic acid by autoxidation. The lack of 7α-hydroxycarnosic acid in incubation mixtures containing Compound 5 rules out autoxidation of carnosic acid and introduction of oxygen from water at position-7.

Compounds or metabolites 4-6 were obtained from preparative-scale incubations of carnosic acid with Nocardia sp. NRRL 5646 after 72 h of incubation in 10.5%, 38% and 8% yields, respectively. Following solvent extraction and column chromatographic purification, samples of metabolites were subjected to spectral analysis.

### EXAMPLES

Isolation of Carnosic Acid. Deodorized rosemary extract (Rosmarinus officinalis L., leaves, 450 g) obtained through an extraction with tetrafluoroethane:acetone:methanol, 8:1:1, w/w/w) was obtained as a dried residue from Kemin Foods, Inc., Des Moines, Iowa. The residue was exhaustively extracted at room temperature with CHCl₃ (3×5L). The combined CHCl₃ extracts were concentrated in vacuo at 30° C. to a brown residue (388 g) that was fractionated by Si gel flash column chromatogeaphy (7.5×110 cm) using in sequence, n-hexane-EtOAc (95:5→30:70). Eleven fractions were combined based on TLC using solvent systems A and B. Fractions containing carnosic acid were rechromatographed by flash column chromatography over Sepralyte C₁₈ using a MeOH:H₂O gradient solvent system (40→70%), followed by Sephadex LH-20 (25-150 μm, Pharmacia Fine Chemical Co.) column eluted with MeOH:CHCl₃ (1:1) to afford carnosic acid (2.14 g) as a yellowish powder. UV, IR, 'H and ^{'3}C NMR, and ESIMS data for [M+H]⁺ ion in good agreement with reported data for carnosic acid (1) (C₂₀H₂₉O₄ [M+H]⁺, 333) (Curvelier, M. E.; Richard, H.; Berset, C. J. Am. Oil Chem. Soc. 1996, 73, 645-652; Schwarz, K.; Ternes, W. Z. Lebensm Unters. Forsch. 1992, 195, 99-103).

Microorganism. Nocardia sp. NRRL 5646 is maintained in the University of Iowa, College of Pharmacy culture collection on slants of Sabouraud-dextrose agar or sporulation agar (ATCC no. 5 medium)(Gherna, R.; Pienta, P.; Cote, R. ATCC Cataloge of Bacteria and Bacteriophages, 18th Ed.; American Type Culture Collection, Rockville, Md., USA 1992, p. 415).

Analytical-Scale Biotransformation. A two-stage fermentation protocol was used for analytical and preparative scale formation of carnosic acid metabolites. The medium contained 0.5% (w/v) soybean meal, 0.5% yeast extract, 0.5% NaCl, 0.5% K₂HPO₄, and 2% dextrose per 1 L of distilled water, adjusted to pH 7.0 with 6 N HCl, was autoclaved at 121° C. for 15 min. Analytical incubations were conducted in 25 mL of sterile medium held in 125 mL stainless steel-capped Delong culture flasks that were incubated with shaking at 250 rpm at 28° C. on a New Brunswick Scientific, Innova 5000 Gyrotory three-tier shaker. A 10% inoculum derived from 72 h old stage I cultures was used to initiate stage II cultures, which were incubated for 24 h more before receiving 5 mg of carnosic acid in 0.2 mL of N,N-dimethylformamide, and incubations were continued. Substrate controls consisted of sterile medium and substrate incubated under the same conditions but without microorganism. Samples of 3 mL were withdrawn for analysis at 24, 48, 72, and 144 h after substrate addition, extracted with 1 mL of EtOAc. The organic layer was separated from aqueous medium by centrifugation at 1,200×g in a desktop centrifuge and 30 μL samples were spotted onto TLC plate developed with solvent system A. On the basis of screening experiments, three metabolites were reproducibly formed by Nocardia with R_{f} values of 0.88 for Compound 4, 0.71 for Compound 5 and 0.54 for Compound 6.

Preparative Biotransformation of Carnosic Acid. Nocardia cultures were incubated as before in ten, 125 mL stainless steel-capped Delong culture flasks, each containing 25 mL of medium. Carnosic acid (100 mg) was dissolved in 1 mL N,N-dimethylformamide and evenly distributed among the 24 h-old stage-II cultures. After 72 h, the contents of 10 flasks were combined and centrifuged at 10,000×g at 4° C. for 20 min. The supernatant (225 mL) was extracted with three 500 mL volumes of EtOAc. The organic layers were pooled, dried over anhydrous Na₂SO₄, filtered through sintered glass, and vacuum-concentrated to yield 93 mg of a viscous brown residue.

Isolation of Metabolites. The brown residue was subjected to Si gel flash column chromatography (1.5×50 cm) using in sequence, Hexane/EtOAC (95:5→75:25). Two fractions, A (22 mg) and B (53 mg), were obtained based on TLC analysis. Fractions A and B were separately resolved by reversed phase, Sepralyte C₁₈ Si gel flash column chromatography (1×50 cm), using a MeOH—H₂O gradient solvent system (40→70%) under column pressures of 0.28 kg/cm², at a flow rate of 2 mL/min while 3 mL fractions were collected. Final sample purifications were carried out with Sephadex LH-20 (25-150 μm. Pharmacia Fine Chemical Co.) columns eluted with MeOH:CHCl₃ (1:1) to afford carnosic acid metabolites Compound 4 (10.5 mg), Compound 5 (38 mg), and Compound 6 (8 mg).

General Experimental Procedures. Optical rotations were measured with a JASCO P-1020 polarimeter. UV spectra were determined with a Hitachi 340 spectrophotometer. IR spectra (cm⁻¹) were obtained using a Nicolet 205 FT-IR spectrometer connected to a Hewlett-Packard ColorPro plotter. High resolution Electro Spray Ionization mass spectra (HRESIMS) were taken on a VG-ZAB-HF reversed geometry (BE configuration, where B is a magnetic sector and E is an electrostatic analyzer) mass spectrometer (MS) (VG Analytical, Inc.).

NMR spectra were obtained in acetone using TMS as the internal standard, with chemical shifts expressed in (δ) and coupling constants (J) in Hz. Routine ¹H and ¹³C NMR spectra were obtained with a Bruker NMR 400 (Bruker Instruments, Billerica, Mass.), operating at 400 MHz (¹H) and 100 MHz (¹³C). DQF-COSY, NOESY, HMQC and HMBC NMR experiments were carried out using a Bruker AMX-600 high-field spectrometer equipped with an IBM Aspect-2000 processor and with software VNMR version 4.1. Flash column liquid chromatography was performed using J. T. Baker glassware with 40-μm silica gel (Baker) and Sepralyte C₁₈ (40 μm) as the stationary phase. TLC was carried out on precoated silica gel 60 F₂₅₄ (Merck) plates. Developed chromatograms were visualized by spraying developed plates with 1% vanillin/H₂SO₄, followed by heating at 100° C. for 3 min. TLC plates were developed with solvent systems: A (EtOAc:Hexane, 1:1, v:v) or B (CHCl₃:MeOH, 8.5:1.5, v:v).

DPPH Radical Scavenging Assay. The DPPH free radical scavenging assay is based on the abilities of compounds to quench stable DPPH free radicals (Lee, S. K.; Mbwambo, Z. H.; Chung, H. S.; Luyengi, L.; Gamez, E. J. C.; Mehta, R. G.; Kinghorn, A. D.; Pezzuto, J. M. Comb. Chem. High Throughput Screen. 1998, 1, 35-46). Reaction mixtures (200 μL) were prepared by combining multiple concentrations of test sample in methanol (10 μL) with DPPH (Aldrich, Milwaukee, Wis.) in methanol (190 μL). The final DPPH concentration was 300 μM. The reaction mixtures were incubated in 96-well microtiter plates at room temperature for 30 min. After the reaction, reduction of the radical was then measured at 517 nm. Percent inhibition by sample treatment was determined by comparison with controls containing no test samples. IC₅₀ values denote the concentration of sample, required to scavenge 50% DPPH free radicals (Ibid.).

### Compound or Metabolite 4

Metabolite 4 was obtained as yellowish crystals (10.5 mg); [α]²⁵_{D}+58.9° (c 0.21, CHCl₃); UV (MeOH) λₘₐₓ (log ε) 205 (4.30), 236 (3.59) nm; IR (KBr) νₘₐₓ 3375 (OH), 1595 and 1515 (aromatic ring) cm⁻¹; ¹H NMR (acetone, 600 MHz) δ 1.72 (1H, dt, J=3.5, 13.5 Hz, H-1α), 3.11 (1H, brd, J=13.5 Hz, H-1, 1.58 (1H, dd, J=6.2, 11.7 Hz, H-2α), 2.94 (1H, ddd, J=5.5, 6.2, 11.7 Hz, H-2β), 1.54 (1H, m, H-3α), 2.09 (1H, m, H-3β), 1.50 (1H, d, J=12.3 Hz, H-5α), 1.73 (1H, dd, J=5.9, 13.0 Hz, H-6α) 1.92 (1H, dq, J=4.8, 13.0 Hz, H-6β), 2.89 (2H, m, H₂-7), 6.88 (1H, s, H-14), 3.09 (1H, sept, J=7.0 Hz, H-15), 1.16 (3H, d,J=7.0 Hz, Me-16), 1.19 (3H, d, J=7.0 Hz, Me-17), 0.86 (3H, s, Me-18), 1.12 (3H, s, Me-19), 3.87 (1H, dd, J=2.7, 11.7 Hz, H-20A), 4.12 (1H, dd, J=6.3, 11.7 Hz, H-20B), 3.86 (3H, s, OCH₃), 6.11 (1H, s, ArOH); ¹³C NMR (acetone, 100 MHz) δ 38.35 t (C-1), 18.73 t (C-2), 36.76 t (C-3) 35.10 s (C-4), 52.18 d (C-5), 20.50 t (C-6), 34.48 t (C-7), 135.45 s (C-8), 128.14 s (C-9), 42.70 s (C-10), 149.68 s (C-11), 152.30 s (C-12), 137.94 s (C-13), 118.00 d (C-14), 27.50 d (C-15), 22.13 q (C-16), 22.76 q (C-17), 21.12 q (C-18), 26.55 q (C-19), 69.22 t (C-20), 56.80 q (OCH₃); HRESIMS m/z [M+H]⁺ 333.2434 (calculated for C₂₁H₃₃O₃, 333.2429).

### Compound or Metabolite 5

Metabolite 5 gave UV, IR, ¹H and ¹³C NMR, and ESIMS data for [M+H]⁺ ion in good agreement with reported data for carnosol (C₂₀H₂₇O₄ [M+H]⁺ 331) (Inatani, R.; Nakatani, N.; Fuwa, H.; Seto, H. Agric. Biol. Chem. 1982,46, 1661-1666).

### Compound or Metabolite 6

Metabolite 6 was obtained as a yellowish amorphous powder, (8 mg); [α]²⁵_{D}+67.2° (c 0.34, CHCl₃); UV (MeOH) λₘₐₓ (log ε) 220 (4.36), 287 (3.78) nm; IR (KBr) νₘₐₓ 3408 (OH), 1750 (γ-lacton), 1590 and 1515 (aromatic ring) cm⁻¹; ¹H NMR (acetone, 600 MHz) δ 2.14 (1H, dt, J=5.1, 13.8 Hz, H-1β), 3.02 (1H, brd, J=13.8 Hz, H-1β), 1.96 (1H, dd, J=5.1, 12.8 Hz, H-2α), 2.64 (1H, dt, J=5.1, 12.8 Hz, H-2β), 1.44 (1H, m, H-3α), 1.82 (1H, m, H-3β), 1.72 (1H, dd, J=5.7, 10.9 Hz, H-5α), 2.68 (1H, ddd, J=4.5, 5.7, 13.5 Hz, H-6α) 1.95 (1H, ddd,J=1.7, 10.9, 13.5 Hz, H-6β), 5.43 (1H, dd, J=1.7, 4.5 Hz, H-7), 6.82 (1H s, H-14), 3.39 (1H, quint., J=7.0 Hz, H-15), 3.86 (1H, dd, J=2.6, 10.8 Hz, H16A), 4.09(111, dd, J=6.3, 10.8 Hz, H-16B), 3.93 (1H, dd, J=2.8, 10.8 Hz, H-17A), 4.12 (1H, dd,J=6.5, 10.8 Hz, H-17B), 1.10 (3H, s, Me-18), 0.95 (3H, s, Me-19), 6.37 (1H, s, ArOH), 6.85 (1H, s, ArOH); ¹³C NMR (acetone, 100 MHz) δ 28.76 t (C-1), 19.62 t (C-2), 42.55 t (C-3) 36.35 s (C-4), 46.58 d (C-5), 30.15 t (C-6), 77.82 d (C-7), 136.30 s (C-8), 128.22 s (C-9), 49.50 s (C-10), 149.11 s (C-11), 150.10 s (C-12), 139.12 s (C-13), 118.15 d (C-14), 28.87 d (C-15), 69.33 t (C-16), 71.14 t (C-17), 22.10 q (C-18), 30.05 q (C-19), 176.52 s (C-20); HRESIMS m/z [M+H]⁺ 363.1811 (calculated for C₂₀H₂₇O₆, 363.1807).

Metabolite or compound 4 gave C₂₁H₃₃O₃ (m/z 333.2434, M+H⁺) by HRESIMS, which indicated the addition of one carbon, the removal of one oxygen, and the reduction of an unsaturated center versus carnosic acid. As with carnosic acid, the ¹H NMR spectrum of Compound 4 showed four methyl-group signals at δ 0.86 (3H, s, Me-18α), δ 1.12 (3H, s, Me-19β), δ 1.12 (3H, d, J=7.0 Hz, Me-16) and δ 1.19 (3H, d, J=7.0 Hz, Me-17) confirming the presence of geminal 18,19-methyl groups, and the isopropyl moiety attached at position 13. The m/z 288 fragment in the MS spectrum represented the loss of the isopropyl group from the molecular ion. Signals for all ring protons and carbons were confirmed by DQF-COSY, HMQC and HMBC spectra. The major differences in Compound 4 versus carnosic acid were in the absence of the C-20 carboxyl group signal, the presence of two double doublets as an AB system at δ 3.87, (1H, dd, J=2.7, 11.7 Hz, H-20A) and δ 4.12 (1H, dd, J=6.3, 11.7 Hz, H-20B), and a 3H singlet at δ 3.86 for a methoxyl group. The hydroxy-methylene group was correlated with a carbon signal at δ 69.22 in the HMQC, and showed long-rang coupling with H-1α by DQF-COSY to suggest that the carboxylic acid group on C-10 of carnosic acid was reduced to an alcohol in Compound 4 by Nocardia sp. The methoxyl group was correlated with the carbon resonance at δ 152.30 (C-12). Irradiation of Compound 4 at δ 3.86 (12-O-methyl protons) caused NOE enhancement in the signal of H-15 at δ 3.09 (8%) and a minor effect on the signals of the isopropyl methyl groups at δ 1.16 and δ 1.19 (2% each). Thus, the methoxyl and the isopropyl groups were ortho to each otherr.

The significant HMBC cross-peaks observed from the hydroxy-methylene protons at δ 69.22 with methylene carbon at δ 38.35 (C-1), methine carbon at δ 52.18 (C-5), and quaternary carbons at δ 128.14 (C-9) and δ 42.70 (C-10) were also consistent with C-20 alcohol. Therefore, the structure of new Compound 4 was 11,20-dihydroxy-12-methoxy-abiet-8,11,13-triene.

Metabolite or Compound 5 gave m/z 331 [M+H]⁺ for C₂₀H₂₇O₄ by ESIMS which indicated a structure with two fewer hydrogens versus carnosic acid, that could be accounted for by a carnosol derivative formed by oxidative cyclization. Spectral data showed that Compound 5 was the same as carnosol (Inatani, R.; Nakatani, N.; Fuwa, H.; Seto, H. Agric. Biol. Chem. 1982,46, 1661-1666) or 11,12-dihydroxy-7-10-(epoxymethano)-abiet-8,11,13-triene-20-one.

Metabolite or Compound 6 gave m/z 331 [M+H]⁺ for C₂₀H₂₇O₆ by HREIMS indicating a structure like Compound 5 plus two additional hydroxyl groups. The ¹H and ¹³C NMR spectra of Compound 6 showed signals with chemical shifts, multiplicities and coupling constant similar to those of Compound 5 except for the replacement of the signals of the isopropyl moiety of Compound 5 with those of a dihydroxylated isopropyl moiety. That is, the ¹H and ¹³C NMR spectra of Compound 6 showed additional signals of two oxygenated methylene groups shielded at high field at δ 3.86 (1H, dd, J=2.6, 10.8 Hz, H-16A), δ 4.09 (1H, dd, J=6.3, 10.8 Hz, H-16B), δ 3.93 (1H, dd, J=2.8, 10.8 Hz, H-17A) and δ 4.12 (1H, dd, J=6.5, 10.8 Hz, H-17B). The C-15 methine proton at δ 3.39 (1H, quintet, J=7.0 Hz, H-15) was shifted downfield relative to that of carnosic acid. Two singlet protons at δ 6.37 and 6.85 (disappeared after addition of D₂O) were assigned to the hydroxy phenol groups.

HMQC and HMBC spectra of Compound 6, as well as the characteristic fragment ion at m/z 286 consistent with loss of a dthydroxylated isopropyl moiety in the ESIMS confirmed the location of the two new hydroxyl groups at positions-16 and -17. Therefore, Compound 6 was identified as the new metabolite, 11,12,16,17-tetrahydroxy-7-10-(epoxymethano)-abiet-8,11,13-triene-20-one.

Antioxidant Properties. To evaluate the three carnosic acid metabolites, Compounds 4-6, for antioxidant potential, their DPPH free radical scavenging activities were compared with those of carnosic acid and other antioxidants including mixed tocopherols, ascorbic acid, pyrogallol and propyl gallate (Table 1). Among the three biotransformation products, carnosol exhibited the lowest activity in DPPH quenching (IC₅₀=37.9 μg/mL). Metabolites 4 (IC₅₀=27.7 μg/mL) and 6 (IC₅₀=23.9 μg/mL) showed slightly less DPPH activity than carnosic acid (IC₅₀=18.7 μg/mL) but slightly more than mixed tocopherols (IC₅₀=28.4 μg/mL). These finding emphasize that the previously unknown metabolites 4 and 6 could be better antioxidants in the radical scavenging process.

TABLE 1
Activities of Metabolites 4-6 in the DPPH
Free-Radical Scavenging Assay

| Sample | IC₅₀ (μg/mL)¹ | IC₅₀ (μM)¹ |
|---|---|---|
| 1 | 18.7 | 53.3 |
| 4 | 27.7 | 83.4 |
| 5 | 37.9 | 114 |
| 6 | 23.9 | 66.0 |
| Mixed tocopherols² | 28.4 | |
| Ascorbic acid | 11.8 | 66.8 |
| Pyrogallol | 4.8 | 37.8 |
| Propyl gallate | 9.8 | 46.1 |

¹Values are presented as the mean of 3-test sample observations.
²Mixed tocopherols consists of a blend of α, δ and γ in an approximate ratio of 1:3:7.

Conversions of carnosic acid to Compounds 4, 5 and 6 by Nocardia provides a reproducible means of affording carnosol (Compound 5) and the two new derivatives of carnosic acid, Compound 4 and Compound 6. Microbial reduction of the carboxylic acid moiety of carnosic acid adds to the wide list of substrates that are reduced by the carboxylic acid reductase and aldehyde reductase enzyme systems of Nocardia (Li, T.; Rosazza, J. P. N. J. Bacteriol. 1997, 179, 3482-3487; Chen, Y.; Rosazza, J. P. N. Appl. Enivron. Microbiol. 1994, 60, 1292-1296; Li, T.; Rosazza, J. P. N. J. Biol. Chem. 1998, 273, 34230-34233). The Nocardia carboxylic acid reductase is a unique enzyme that binds carboxylic acids, ATP and NADPH. The first intermediate produced is a carbonyl-activated acyl-adenylate derivative that is reduced by hydride delivery from NADPH to afford aldehydes. In whole cell Nocardia cultures, a separate NADPH-dependent, alcohol oxidoreductase reduces aldehydes to the corresponding alcohols (Compound 3 of FIG. 1). The methoxyl group at position-12 in Compound 6 is likely introduced by means of an S-adenosylmethionine-dependent, catechol-O-methyl transferase (COMT) system similar to that recently characterized in Streptomyces griseus (Dhar, K.; Rosazza, J. N. P. Appl. Enivron. Microbiol. 2000, 66, 4877-4882). The conversion of carnosic acid (1) to carnosol (5) likely involves enzymatic oxidation of carnosic acid to a quinoid intermediate followed by intramolecular Michael addition of the carboxylate anion at position-7. Subsequent hydroxylations of Compound 5 to Compound 6 are likely catalyzed by unknown enzymes.

Although the invention has been described with respect to a preferred embodiment thereof, it is to be also understood that it is not to be so limited since changes and modifications can be made therein which are within the full intended scope of this invention as defined by the appended claims.

We claim:

1. Compounds having the structure comprising: 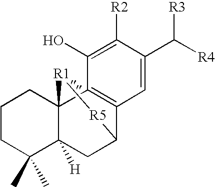
wherein, R1, R2, R3, R4, and R5 are selected from the group consisting of —C═O, —OH, —CH₂OH, —CH₂OH, and —O—, respectively, and —CH₂OH, —OCH₃, —CH₃, —CH₃, and absent, respectively.

2. A compound as defined in claim 1 having the name 11,20-dihydroxy-12-methoxy-abiet-8,11,13-triene.

3. A compound as defined in claim 1 having the name 11,12,16,17-tetrahydroxy-7-10-(epoxymethano)-abiet-8,11,13-triene-20-one.

4. A compound as defined in claim 1 comprising a metabolite of carnosic acid.

5. A compound as defined in claim 4, wherein the compound is a metabolite of carnosic acid by Nocardia sp.

6. A metabolite of carnosic acid by bacteria having antioxidant properties greater than that of carnosic acid.

7. A derivative of carnosic acid, comprising reduction of the C-20 carboxylic acid functional group and methylation at the C-12 phenol moiety.

8. A derivative of carnosic acid, comprising oxidative cyclization of carnosic acid to carnosol and dihydroxylation at the isopropyl moiety.

9. A derivative of carnosol, comprising dihydroxylation at the isopropyl moiety.

10. A compound as defined in claim 1 comprising a metabolite of carnosol.

11. A compound as defined in claim 4, wherein the compound is a metabolite of carnosol by Nocardia sp.

12. A metabolite of carnosol by bacteria having antioxidant properties greater than that of carnosic acid.
